(19) United States
(12) Patent Application Publication
(10) Pub. No.: US 2010/0249509 A1
(43) Pub. Date: Sep. 30, 2010

(54) INTRAVITAL OBSERVATION SYSTEM AND METHOD OF DRIVING INTRAVITAL OBSERVATION SYSTEM

(75) Inventor: Fukashi YOSHIZAWA, Ina-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/730,589

(22) Filed: Mar. 24, 2010

(30) Foreign Application Priority Data
Mar. 30, 2009 (JP) 2009-083211

(51) Int. Cl. A61B 1/00

(52) U.S. Cl. 600/118

(57) ABSTRACT

The intravital observation apparatus according to the present invention is provided with a capsule endoscope including an illumination section and an image pickup section, a power supply section provided with a battery and a control section, and a control signal generation apparatus disposed outside the capsule endoscope, wherein the control signal generation apparatus is provided with a control signal generation section and a control signal transmitting electrode, and the capsule endoscope is provided with a control signal receiving electrode and a control signal detection section.

This application claims benefit of Japanese Application No. 2009-083211 filed in Japan on Mar. 30, 2009, the contents of which are incorporate by this reference.

### BACKGROUND OF THE INVENTION

1. Field of the Invention

The present invention relates to an intravital observation apparatus, an intravital observation system including a control signal generation apparatus disposed outside the intravital observation apparatus and a method of driving the intravital observation system.

2. Description of the Related Art

In recent years, an ultra-compact endoscope in which an image pickup unit and an illumination optical system or the like are accommodated, for example, in a tablet capsular casing, or so-called “capsule endoscope” is being developed as an intravital observation apparatus.

A capsule endoscope is introduced into a body cavity by means of swallowing by an examinee and used to pick up an image of a diseased part and transmit the image to the outside of the body. By receiving this transmitted image outside the body, it is possible to observe or inspect the interior of the body cavity. Therefore, the capsule endoscope has an advantage of being able to relatively easily perform an observation or inspection or the like of an organ such as the small intestine, which has been hard to be observed or inspected using a conventional endoscope having an insertion portion.

Furthermore, a method using a magnet is known as the method of controlling activation and stopping of the capsule endoscope, to be more specific, controlling starting and stopping of image pickup or starting and stopping of illumination in a non-contact manner from outside, and such a method is disclosed in Japanese Patent Application Laid-Open Publication No. 2001-224553.

Japanese Patent Application Laid-Open Publication No. 2001-224553 discloses a configuration in which a reed switch is used as a power switch to turn ON/OFF a power supply from a battery provided inside the capsule endoscope to each member of the capsule endoscope.

The reed switch provided inside the capsule endoscope disclosed in Japanese Patent Application Laid-Open Publication No. 2001-224553 is configured so that power is switched ON/OFF in a non-contact manner depending on the presence/absence of a magnetic field, and a contact is opened when the reed switch is placed in the magnetic field and the power supply is stopped.

That is, the reed switch is configured such that the reed switch is turned OFF when the capsule endoscope is accommodated in a container box or case provided with a magnet, the power supply in the capsule endoscope is interrupted and the capsule endoscope is stopped, whereas the reed switch is turned ON when the capsule endoscope is taken out of the container box or case, the power is supplied to the capsule endoscope and the capsule endoscope is activated.

### SUMMARY OF THE INVENTION

Briefly, the intravital observation system of the present invention is provided with an intravital observation apparatus including an intravital information acquisition section that acquires at least intravital information and a power supply section provided with a battery that supplies drive power to the intravital information acquisition section and a control section that controls supply or interruption of the drive power supplied from the battery, and a control signal generation apparatus disposed outside the intravital observation apparatus for activating or stopping the intravital observation apparatus, wherein the control signal generation apparatus is provided with a generation section that generates a control signal for activating or stopping the intravital observation apparatus and a control signal transmitting electrode that transmits the control signal, and the intravital observation apparatus is provided with a control signal receiving electrode that receives the control signal transmitted from the control signal transmitting electrode and a control signal detection section that detects the control signal inputted via the control signal receiving electrode and controls power supply/interruption operation of the control section.

The above and other objects, features and advantages of the invention will become more clearly understood from the following description referring to the accompanying drawings.

### BRIEF DESCRIPTION OF THE DRAWINGS

FIG. 1 is a diagram schematically illustrating a configuration of an intravital observation system illustrating a first embodiment;

FIG. 2 is a diagram schematically illustrating a configuration of the capsule endoscope in FIG. 1;

FIG. 3 is a diagram schematically illustrating a configuration of the electric circuit of the capsule endoscope in FIG. 2;

FIG. 4A is a timing chart illustrating an AC signal generated from the control signal generation apparatus;

FIG. 4B is a timing chart illustrating signal output of the control signal detection section of the capsule endoscope;

FIG. 4C is a timing chart illustrating signal output of an inverter inputted to the gate of a P-channel type FET of the power supply section;

FIG. 4D is a timing chart illustrating a power supply state of the capsule endoscope;

FIG. 5 is a diagram schematically illustrating a modification example of the configuration of the electric circuit of the control signal detection section of the capsule endoscope in FIG. 3;

FIG. 6 is a diagram schematically illustrating a modification example of a configuration of an electric circuit of a capsule endoscope of an intravital observation system illustrating a second embodiment;

FIG. 7A is a timing chart illustrating an AC signal generated from the control signal generation apparatus;

FIG. 7B is a timing chart illustrating signal output of the control signal detection section of the capsule endoscope;

FIG. 7C is a timing chart illustrating signal output of a frequency dividing circuit inputted to the gate of the P-channel type FET of the power supply section; and

FIG. 7D is a timing chart illustrating a power supply state of the capsule endoscope.

### DETAILED DESCRIPTION OF THE PREFERRED EMBODIMENTS

Hereinafter, embodiments of the present invention will be described with reference to the accompanying drawings. It should be noted that the drawings are schematic ones and the relationship between thickness and width of each member, ratio of thickness among the respective members or the like are different from the actual ones, and it goes without saying that parts are also included which differ in the dimensional relationship and ratio among the drawings.

Furthermore, the intravital observation apparatus will be described in the following embodiments by taking a capsule endoscope as an example. A general capsule endoscope is provided with an illumination section, an image pickup section, a wireless transmission section that wirelessly transmits image data obtained by the image pickup section and a power supply section that supplies drive power to these members, and can observe internal situations of organs such as the esophagus, stomach, small intestine and large intestine.

### First Embodiment

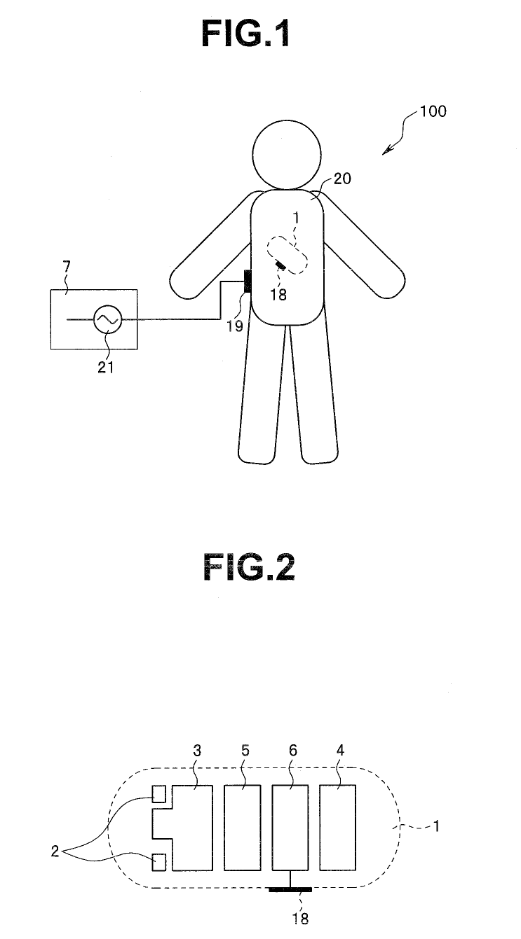

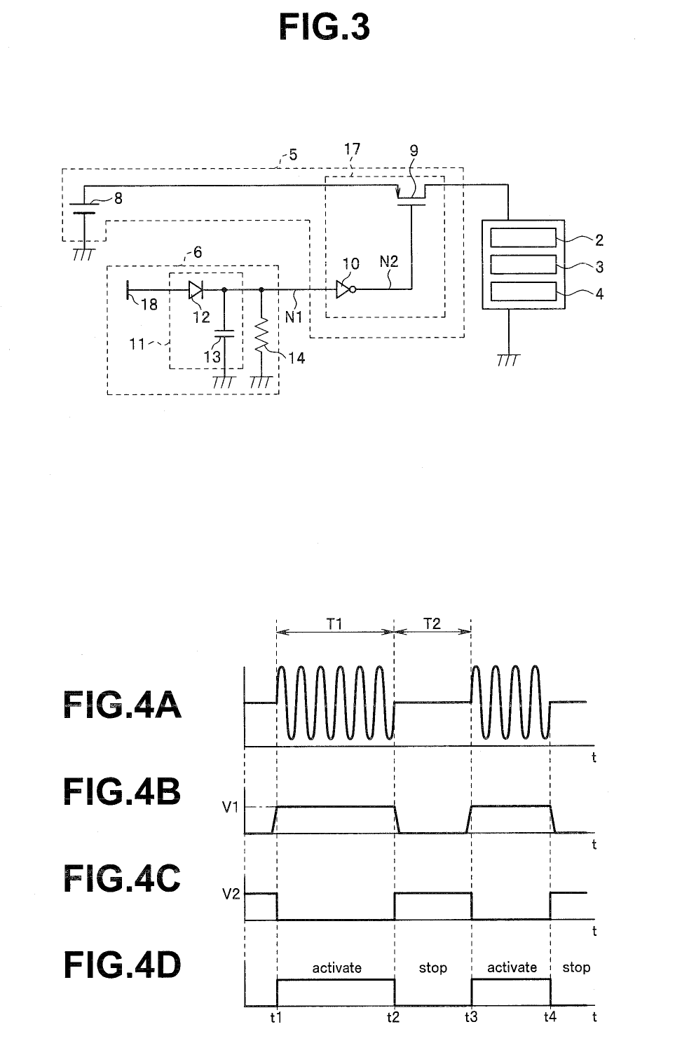

FIG. 1 is a diagram schematically illustrating a configuration of an intravital observation system illustrating the present embodiment, FIG. 2 is a diagram schematically illustrating a configuration of the capsule endoscope in FIG. 1 and FIG. 3 is a diagram schematically illustrating a configuration of the electric circuit of the capsule endoscope in FIG. 2.

Furthermore, FIG. 4A is a timing chart illustrating an AC signal generated from the control signal generation apparatus, FIG. 4B is a timing chart illustrating signal output of the control signal detection section of the capsule endoscope, FIG. 4C is a timing chart illustrating signal output of an inverter inputted to the gate of P-channel type FET of the power supply section and FIG. 4D is a timing chart illustrating a power supply state of the capsule endoscope.

As shown in FIG. 1, main parts of an intravital observation system 100 are made up of a capsule endoscope 1 and a control signal generation apparatus 7 disposed outside the capsule endoscope 1 to start or stop the capsule endoscope 1.

As shown in FIG. 2, main parts of the capsule endoscope 1 are made up of an illumination section 2, which is an intravital information acquisition section that acquires at least intravital information, an image pickup section 3, which is an intravital information acquisition section, a wireless transmission section 4, a power supply section 5 and a control signal detection section 6.

The illumination section 2 illuminates an observed region after the capsule endoscope 1 is started and the image pickup section 3 picks up an image of the observed region after the capsule endoscope 1 is started.

The wireless transmission section 4 wirelessly transmits an image pickup signal, which is living body information of the image picked up by the image pickup section 3, to outside of a living body, for example, a receiving apparatus (not shown), and the power supply section 5 supplies drive power to the illumination section 2, the image pickup section 3 and the wireless transmission section 4.

As shown in FIG. 3, main parts of the power supply section 5 are made up of a battery 8 and a control section 17 that controls supply or interruption of the drive power supplied from the battery 8.

Main parts of the control section 17 are made up of an inverter 10 that inverts the output from the control signal detection section 6 and a P-channel type FET 9, which is a switching element, the source of which is connected to the battery 8, the gate of which is connected to the output of the inverter 10 and the drain of which is connected to the circuits such as the illumination section 2, the image pickup section 3 and the wireless transmission section 4.

Since the FET 9 controls starting and stopping of a supply of drive power from the battery 8, the control section 17 controls starting or stopping of the power supply from the battery 8 to each circuit in the capsule endoscope 1.

As shown in FIG. 3, main parts of the control signal detection section 6 are made up of a control signal receiving electrode 18, a rectification circuit 11 made up of a diode 12 and a smoothing capacitor 13 connected to the control signal receiving electrode 18 and a resistor 14 connected parallel to the smoothing capacitor 13.

The control signal detection section 6 detects an AC signal which is a control signal transmitted from the control signal generation apparatus 7 via the control signal receiving electrode 18, generates a control signal for control continuity or non-continuity of the FET 9 in the power supply section 5, and thereby controls power supply/interruption operation in the control section 17 of the power supply section 5.

The control signal receiving electrode 18 receives an AC signal transmitted from a control signal transmitting electrode 19 (see FIG. 1), which will be described later, of the control signal generation apparatus 7, is disposed on the outer surface of the capsule endoscope 1 and configured to be able to contact the living body directly or via an insulating member.

To be more specific, when the control signal receiving electrode 18 is disposed so as to be exposed out of the outer surface of the capsule endoscope 1, the control signal receiving electrode 18 can directly contact the living body.

Alternatively, when the control signal receiving electrode 18 is covered with an insulating member made of resin of the same material as the casing making up the outer surface of the capsule endoscope 1, the control signal receiving electrode 18 can contact the living body via the insulating member.

Furthermore, a metal material having less biological reaction such as stainless steel, cobalt-chromium alloy, titanium, titanium alloy, gold or platinum is preferable as the material making up the control signal receiving electrode 18.

The control signal detection section 6 can operate without consuming power of the battery 8, and therefore if the inverter 10 is made up of CMOS or the like, it is possible to reduce power consumption to substantially 0 when the capsule endoscope 1 is in a stopped state.

As shown in FIG. 1, main parts of the control signal generation apparatus 7 are configured by including an AC signal generation section 21 for starting or stopping the capsule endoscope 1 and the control signal transmitting electrode 19 for transmitting an AC signal into the living body.

The control signal transmitting electrode 19 may be directly attached to the surface of the living body 20 or attached to the surface of the living body 20 placed in a case made of an insulating member such as resin, in other words, with an insulating member interposed in between. Furthermore, the control signal transmitting electrode 19 may be attached over clothing made of an insulating member.

An AC signal for starting or stopping the capsule endoscope 1 generated from the generation section 21 of the control signal generation apparatus 7 is applied to the living body (examinee) via the control signal transmitting electrode 19.

The AC signal applied to the living body (examinee) is transmitted to the aforementioned control signal receiving electrode 18 provided in the capsule endoscope 1 via an impedance of the living body. To be more specific, the AC signal is transmitted to the control signal receiving electrode 18 via cells such as fat, muscles, bone, mucous membrane or blood, digestive juice or the like of the living body.

Here, when the control signal transmitting electrode 19 is attached to the surface of the living body 20 with the insulating member interposed in between, the AC signal is applied and transmitted to the living body through capacitative coupling with the living body. Furthermore, the region in which the control signal transmitting electrode 19 is attached is not particularly limited because there is no directivity in the AC signal that transmits through the living body.

Furthermore, when the control signal receiving electrode 18 provided for the capsule endoscope 1 is provided on the outer surface of the capsule endoscope 1, the AC signal transmitted through the living body is directly transmitted to the control signal receiving electrode 18.

Furthermore, when the control signal receiving electrode 18 is configured to contact the living body via the insulating member, the AC signal is transmitted from the living body to the control signal receiving electrode 18 through capacitative coupling with the living body.

Next, operation of the present embodiment will be described.

Suppose the control signal generation apparatus 7 is placed outside the living body to generate an AC signal as shown in FIG. 1.

First, as shown in FIG. 4A, when an AC signal is generated from the generation section 21 of the control signal generation apparatus 7 at time t1, the AC signal is transmitted from the control signal transmitting electrode 19 to the control signal receiving electrode 18 provided in the capsule endoscope 1 through the living body.

The AC signal transmitted to the control signal receiving electrode 18 is converted to a DC voltage by the rectification circuit 11 made up of the diode 12 and the smoothing capacitor 13, and the potential (V1) at node N1 becomes high level as shown in FIG. 4B.

Thus, as shown in FIG. 4C, the output (potential (V2) at node N2) of the inverter 10 in the power supply section 5 becomes low level and the P-channel type FET 9 turns ON. Therefore, power supply to the illumination section 2, the image pickup section 3 and the wireless transmission section 4 is started. That is, as shown in FIG. 4D, the capsule endoscope 1 is activated.

When the generation of the AC signal from the generation section 21 of the control signal generation apparatus 7 is stopped at time t2, the charge charged in the smoothing capacitor 13 is discharged through the resistor 14 and the potential (V1) at N1 becomes low level as shown in FIG. 4B.

Therefore, as shown in FIG. 4C, the output (potential (V2) at node N2) of the inverter 10 in the power supply section 5 becomes high level, the P-channel type FET 9 turns OFF, the power supply to the illumination section 2, the image pickup section 3 and the wireless transmission section 4 is stopped and the capsule endoscope 1 is stopped as shown in FIG. 4D.

When an AC signal is generated again from the generation section 21 of the control signal generation apparatus 7 at time t3, a power supply to the capsule endoscope 1 is resumed as described above and when the generation of the AC signal is stopped at time t4, the power supply to the capsule endoscope 1 is stopped as described above and the same operation will be repeated hereinafter.

Thus, for a period T1 during which an AC signal is generated from the generation section 21 of the control signal generation apparatus 7, the control section 17 of the control signal detection section 6 controls the power supply from the battery 8 and the capsule endoscope 1 is operating and for a period T2 during which no AC signal is generated, the capsule endoscope 1 is stopped.

Next, a diagnosis or observation method using the capsule endoscope 1 that controls starting and stopping of the power supply using such an AC signal will be described briefly.

First, the control signal transmitting electrode 19 of the control signal generation apparatus 7 is attached to the surface of the body of the examinee directly or with an insulating member interposed in between. The capsule endoscope 1 accommodated in the case not requiring any magnet is extracted from the case and the examinee contains the capsule endoscope 1 in his/her mouth.

The capsule endoscope 1 is activated by generating the AC signal from the generation section 21 of the control signal generation apparatus 7. The examinee then swallows the capsule endoscope 1 contained in the mouth.

When the examinee swallows the capsule endoscope 1 in the active state, an observation or diagnosis inside the esophagus is started after the swallowing. Furthermore, in the case where no observation or diagnosis inside the esophagus is necessary, the capsule endoscope 1 may be activated after the swallowing by generating the AC signal from the generation section 21 of the control signal generation apparatus 7.

Furthermore, it is also possible to activate the capsule endoscope 1 outside the body by the examinee touching the control signal receiving electrode 18 provided in the capsule endoscope 1 by a finger or the like thereby causing the AC signal to be generated.

When an observation or diagnosis is started once, the capsule endoscope 1 may be activated as is or the activation and stopping of the capsule endoscope 1 may be freely controlled by generating or stopping the AC signal from the control signal generation apparatus 7 as described in the present embodiment.

For example, for a period during which the capsule endoscope 1 is passing through a region where no observation is necessary, the operation of the capsule endoscope 1 may be stopped and when the capsule endoscope 1 reaches a desired region, an observation or diagnosis may be conducted by generating the AC signal from the control signal generation apparatus 7 and activating the capsule endoscope 1.

Thus, the present embodiment has presented a case where the operation of the capsule endoscope 1 is stopped for a period during which the capsule endoscope 1 is passing through a region where no observation is necessary after swallowing the capsule endoscope 1 and when the capsule endoscope 1 reaches a desired region, the capsule endoscope 1 is activated by generating an AC signal from the generation section 21 of the control signal generation apparatus 7.

This makes it possible to prevent consumption of the battery 8 and reliably conduct an observation or diagnosis of a desired region and improvement in diagnostic performance can be expected, and therefore it is possible to easily and freely control activating and stopping of the capsule endoscope 1 using quite a simple method and thereby provide an intravital observation system capable of reducing consumption of the battery 8 to a minimum and improving diagnostic performance.

The present embodiment uses a half-wave rectification circuit as the smoothing circuit, but it goes without saying that similar operation can also be realized using a full-wave rectification circuit. Furthermore, when there is a possibility that an error may occur in the circuit operation due to an increase in the potential at node N1, a limiter circuit may be added to place a limit to the potential at node N1.

Furthermore, a P-channel type FET is used as the switching means, but the present invention is not limited thereto and any other electronic switch may be used if such an electronic switch has similar functions.

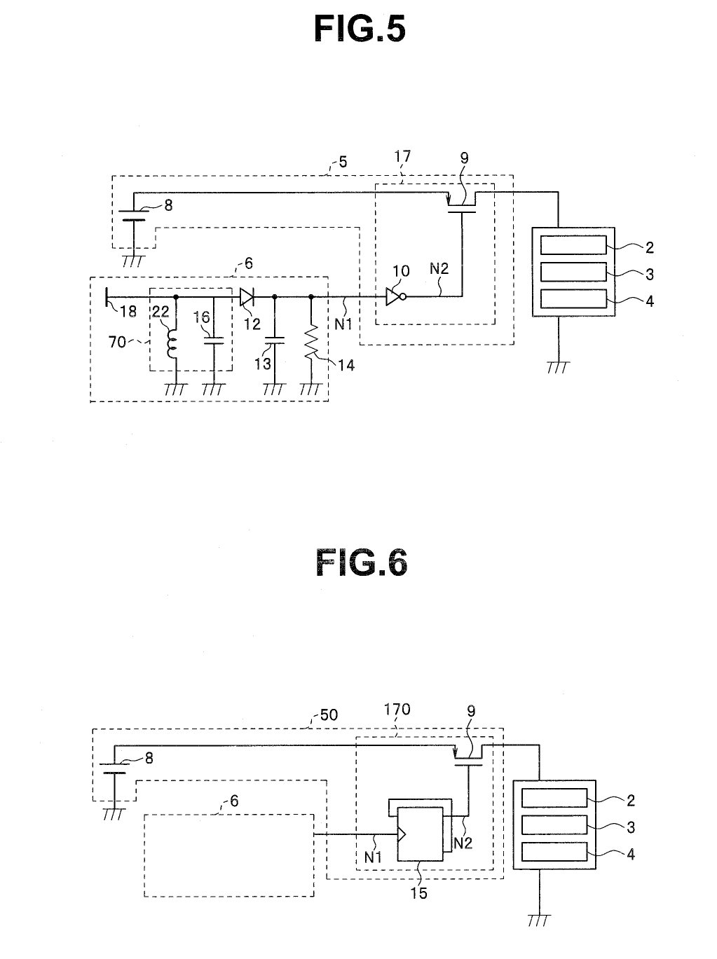

Next, a modification example of the present embodiment will be illustrated using FIG. 5. FIG. 5 is a diagram schematically illustrating a modification example of the configuration of the electric circuit of the control signal detection section of the capsule endoscope in FIG. 3.

As shown in FIG. 5, the control signal detection section 6 is provided with a band-pass filter 70 made up of a coil 22 and a resonance capacitor 16 having a passing frequency equal to the frequency of an AC signal from outside, in addition to the configuration shown in FIG. 3. The rest of the configuration is the same as the configuration shown in FIG. 3.

Stable control over the capsule endoscope 1 without erroneous activation is made possible by causing the passing frequency of the band-pass filter 70 to match the frequency of the AC signal generated from the generation section 21 of the control signal generation apparatus 7.

That is, for an AC signal generated from the control signal generation apparatus 7, detection sensitivity is improved and activation of the capsule endoscope 1 is thereby made easily controllable, whereas for an unintended disturbing signal, the detection sensitivity decreases and erroneous activation can thereby be prevented.

Furthermore, any type of coil can be used for the coil 22, whether a solenoid type coil, a planar coil or the like, and no restriction is imposed on the shape thereof.

### Second Embodiment

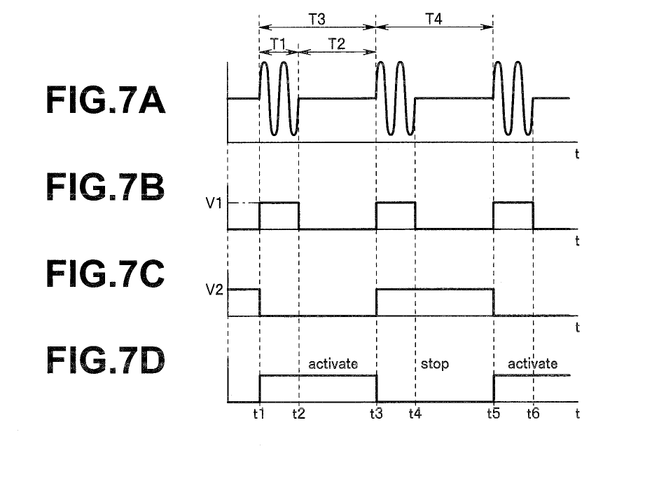

FIG. 6 is a diagram schematically illustrating a modification example of the configuration of the electric circuit of the capsule endoscope of the intravital observation system illustrating the present embodiment, FIG. 7A is a timing chart illustrating an AC signal generated from the control signal generation apparatus, FIG. 7B is a timing chart illustrating signal output of the control signal detection section of the capsule endoscope, FIG. 7C is a timing chart illustrating signal output of a frequency dividing circuit inputted to the gate of a P-channel type FET of the power supply section and FIG. 7D is a timing chart illustrating a power supply state of the capsule endoscope.

The configuration of the intravital observation system of the present second embodiment is different from the intravital observation system of the aforementioned first embodiment shown in FIG. 1 to FIG. 4 in that the control section of the power supply section of the capsule endoscope is provided with a frequency dividing circuit instead of the inverter. Therefore, only this difference will be described and components similar to those of the first embodiment will be assigned the same reference numerals and descriptions thereof will be omitted.

As shown in FIG. 6, main parts of a power supply section 50 are made up of the battery 8, a frequency dividing circuit 15 that divides an output signal (detection signal) from the control signal detection section 6 into two portions and the P-channel type FET 9, the source of which is connected to the battery 8, the gate of which is connected to the output of the frequency dividing circuit 15 and the drain of which is connected to the circuits such as the illumination section 2, the image pickup section 3 and the wireless transmission section 4.

Furthermore, the frequency dividing circuit 15 and the FET 9 constitute a control section 170 that controls the supply/interruption of drive power supplied from the battery 8. The rest of the configuration of the intravital observation system is similar to the configuration of the aforementioned first embodiment.

Next, the operation of the present embodiment will be described.

First, as shown in FIG. 7A, when an AC signal is generated from the generation section 21 of the control signal generation apparatus 7 at time t1, the AC signal is transmitted from the control signal transmitting electrode 19 to the control signal receiving electrode 1R provided in the capsule endoscope 1 through the living body, and the output potential (potential (V1) at node N1) of the control signal detection section 6 becomes high level as shown in FIG. 7B as in the case of the first embodiment.

Next, when the generation of the AC signal from the generation section 21 of the control signal generation apparatus 7 is stopped at time t2, the potential (V1) at the output (node N1) of the control signal detection section 6 becomes low level as shown in FIG. 7B as in the case of the first embodiment.

Hereinafter, the output of the control signal detection section 6 likewise becomes high level for a period T1 during which the AC signal is generated from the control signal generation apparatus 7 and becomes low level for a period T2 during which no AC signal is generated.

As shown in FIG. 7C, the output (node N2) of the frequency dividing circuit 15 of the power supply section 50 becomes low level from time t1 to t3 (period T3) and high level from t3 to t5 (period T4) due to the output signal of the control signal detection section 6.

Therefore, the P-channel type FET 9, the gate of which receives the output signal of the frequency dividing circuit 15, turns ON from time t1 to t3 (period T3) and turns OFF from t3 to t5 (period T4). Therefore, as shown in FIG. 7D, power is supplied from the battery to each circuit of the capsule endoscope 1 for the period T3 and the power supply is stopped for the period T4.

That is, every time an AC signal is generated from the control signal generation apparatus 7 for an extremely short time, the control section 170 repeatedly controls starting or stopping of power supply from the battery 8, in other words, the control section 170 controls starting or stopping of the power supply from the battery 8 through an intermittent AC signal generated from the control signal generation apparatus 7. This allows state control from a stopped state to an activated state and from an activated state to a stopped state of the capsule endoscope 1.

A diagnosis or observation method using the capsule endoscope 1 according to the present embodiment is similar to that of the aforementioned first embodiment, and therefore descriptions thereof will be omitted.

Thus, the present embodiment has shown that application of an AC signal from the control signal generation apparatus 7 for an extremely short time allows control over activation and stopping of the capsule endoscope 1.

This also allows effects similar to those of the first embodiment to be obtained. Furthermore, since the duration during which the AC signal is generated is shorter than that of the first embodiment, it is possible to reduce power consumption of the control signal generation apparatus 7 and the control signal generation apparatus 7 needs to be placed in the vicinity of the examinee only when the capsule endoscope 1 is activated or stopped, and it is thereby possible to reduce burden on not only the operator but also the examinee.

Furthermore, in the present embodiment, the control signal detection section 6 may also be provided with the band-pass filter 70 as shown in FIG. 5. By causing the passing frequency of the band-pass filter 70 to match the frequency of the AC signal generated from the control signal generation apparatus 7, it is possible to improve detection sensitivity with respect to the AC signal generated from the control signal generation apparatus 7, easily control activation and stopping of the capsule endoscope 1 and decrease detection sensitivity for an unintended disturbing signal so as to prevent erroneous activation and erroneous stopping.

The intravital observation apparatus has been described in the aforementioned first and second embodiments by taking the capsule endoscope 1 as an example thereof, but the present invention is not limited thereto, and it goes without saying that effects similar to those of the present embodiments can be obtained also when the present invention is applied to a pH measuring medical capsule or a temperature measuring medical capsule or the like.

Furthermore, the above described embodiments include inventions in various stages and various inventions can be extracted with an appropriate combination of the plurality of components disclosed. For example, even if some components are deleted from all the components shown in one of the above described embodiments, if the problems described in this specification can be solved and effects described herein can be obtained, the configuration with these deleted components can be extracted as an invention.

For example, even if some components are deleted from all the components shown in the above described embodiments, if the problems described in this specification can be solved and effects described herein can be obtained, the configuration with these deleted components can be extracted as an invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An intravital observation system comprising:
an intravital observation apparatus comprising an intravital information acquisition section that acquires at least intravital information and a power supply section provided with a battery that supplies drive power to the intravital information acquisition section and a control section that controls supply or interruption of drive power supplied from the battery; and
a control signal generation apparatus disposed outside the intravital observation apparatus for activating or stopping the intravital observation apparatus,
wherein the control signal generation apparatus comprises a generation section that generates a control signal for activating or stopping the intravital observation apparatus and a control signal transmitting electrode that transmits the control signal, and
the intravital observation apparatus comprises a control signal receiving electrode that receives the control signal transmitted from the control signal transmitting electrode and a control signal detection section that detects the control signal inputted via the control signal receiving electrode and controls power supply/interruption operation of the control section.

2. The intravital observation system according to claim 1, wherein the control section comprises a switching element that controls starting and stopping of supply of the drive power supplied from the battery.

3. The intravital observation system according to claim 1, wherein the control signal is an AC signal generated from the control signal generation apparatus, and
the control signal detection section detects the AC signal and controls power supply/interruption operation of the control section.

4. The intravital observation system according to claim 3, wherein the control signal detection section generates a control signal for controlling continuity or non-continuity of the switching element.

5. The intravital observation system according to claim 1, wherein the control signal detection section comprises a band-pass filter having a passing frequency equal to a frequency of the control signal from outside.

6. The intravital observation system according to claim 1, wherein the control signal transmitting electrode is attached to a living body surface directly or via an insulating member.

7. The intravital observation system according to claim 1, wherein the control signal receiving electrode is disposed on an outer surface of the intravital observation apparatus and is configured so as to be able to contact a living body directly or via an insulating member.

8. The intravital observation system according to claim 1, wherein the intravital observation apparatus is a capsule endoscope.

9. A method of driving an intravital observation system, wherein a control section, which controls a battery that supplies drive power to an intravital information acquisition section that acquires at least intravital information and supply/interruption of the drive power supplied from the battery, controls starting or stopping of a power supply to the intravital information acquisition section by applying an AC signal constituting an intermittent control signal generated from a control signal generation apparatus disposed outside an intravital observation apparatus for activating or stopping the intravital observation apparatus to the intravital observation apparatus.

10. A method of driving an intravital observation system, wherein a control section that controls a battery, which supplies drive power to an intravital information acquisition section that acquires at least intravital information and supply/interruption of the drive power supplied from the battery, controls a power supply to the intravital information acquisition section only for a period during which an AC signal constituting a control signal generated from a control signal generation apparatus disposed outside an intravital observation apparatus for activating or stopping the intravital observation apparatus is applied to the intravital observation apparatus.

11. A method of driving an intravital observation system, wherein a control section, which controls a battery that supplies drive power to an intravital information acquisition section that acquires at least intravital information and supply/interruption of the drive power supplied from the battery, repeatedly controls starting or stopping of a power supply to the intravital information acquisition section every time an AC signal constituting a control signal generated from a control signal generation apparatus disposed outside an intravital observation apparatus for activating or stopping the intravital observation apparatus is applied to the intravital observation apparatus.

12. The method of driving an intravital observation system according to claim 9, wherein the intravital observation apparatus is a capsule endoscope.

13. The method of driving an intravital observation system according to claim 10, wherein the intravital observation apparatus is a capsule endoscope.

14. The method of driving an intravital observation system according to claim 11, wherein the intravital observation apparatus is a capsule endoscope.
